(19) United States
(12) Patent Application Publication
(10) Pub. No.: US 2026/0306784 A1
(43) Pub. Date: Oct. 8, 2026

(54) WEARABLE DEVICE AND SYSTEM FOR ALLEVIATING FATIGUE

(71) Applicant: Jianlin ZHU, Wugang (CN)

(72) Inventor: Jianlin ZHU, Wugang (CN)

(21) Appl. No.: 19/187,907

(22) Filed: Apr. 23, 2025

(30) Foreign Application Priority Data
Apr. 8, 2025 (CN) 202520651678.X

(51) Int. Cl. A61M 21/00; A61H 23/02; A61N 1/38

(52) U.S. Cl. CPC A61M 21/00; A61H 23/02; A61H 2205/067; A61M 2021/0022; A61M 2021/0072; A61M 2021/0083; A61M 2205/3561; A61M 2205/50; A61M 2209/088; A61N 1/38

(57) ABSTRACT

Disclosed are a wearable device and system for alleviating fatigue. The device connected to a terminal device includes: a housing, having an inner surface forming a hollow channel; a power supply assembly, integrated inside the housing, for supplying power for the device's operation; two or more vibration electrodes, disposed on the inner surface, for receiving the power from the power supply assembly to deliver vibrations or electric shocks to the user's skin; and a controller, coupled with the power supply assembly, for controlling the power supply assembly to output electrical signals with preset parameters based on one or more selected modes.

### CROSS-REFERENCE TO RELATED APPLICATIONS

This application claims priority to Chinese Patent Application No. 202520651678.X, filed on Apr. 8, 2025, the content of which is incorporated herein by reference in its entirety.

### TECHNICAL FIELD

The present application relates to a wearable device and a system for alleviating fatigue.

### BACKGROUND

During study and work, people feeling fatigue are becoming more common which leads to difficulty in concentration, difficulty in memorizing new knowledge, and reduce challenge in task completion. The fatigue can even cause people in a slower reaction, a delay in thinking and decision-making processes, and a loss of interest in learning and working, ultimately diminishing human motivation. Most importantly, the fatigue driving is one of the common reason why the traffic accidents occur, the fatigue may severely influence a driver's reaction speed, attention, and judgment especially in a long-time driving. According to the newest research statistics, the fatigue-related driving accidents are directly accounting for 21% annually in China, with a death rate more than 83% in such fatigue-related accidents.

Therefore, to enhance learning and working efficiency, as well as reduce the risk of driving accidents, various different methods are applied to prevent and alleviate human fatigue. One of the methods is to use wearable devices for alleviating fatigue, the wearable device basically generates non-invasive stimulation on the user' skin, as a direct and effective way to alarm and remind the user.

The above content is only used to assist in understanding the technical solution of the present application and does not mean that the above content is recognized as prior art.

### SUMMARY

The main purpose of the present application is to provide a wearable device and system for alleviating fatigue.

In a first aspect, in order to achieve the above objective, the present disclosure to provide a wearable device for alleviating fatigue, connected by communication to a terminal device, includes: a housing, having an inner surface forming a hollow channel; a power supply assembly, integrated inside the housing for supplying power required for operation of the device; two or more vibration electrodes, disposed on the inner surface, configured for receiving the power from the power supply assembly to deliver vibrations or electric shocks to the user for alleviating fatigue; a controller, coupled with the power supply assembly, for controlling the power supply assembly to output electrical signals with preset parameters based on one or more selected modes.

In an embodiment, the power supply assembly includes: a low-frequency signal generator, configured for providing low-frequency pulse currents to the vibration electrodes; and a modulation part, coupled with the low-frequency signal generator, for modulating at least one of waveform, pulse width, current, or voltage of the low-frequency pulse currents generated by the low-frequency signal generator; wherein the controller is further configured to, in response to one or more selected modes, control the modulation part to output the low-frequency pulse currents with the preset parameters to the vibration electrodes.

In an embodiment, the power supply assembly includes a vibration motor, configured for providing mechanical vibrations to the vibration electrodes; the controller is further configured to, in response to one or more selected modes, control the vibration motor to output the mechanical vibrations to the vibration electrodes.

In an embodiment, the controller further includes a first button and a second button protruding from an outer surface of the housing; the first button and the second button are oppositely disposed on the outer surface of the housing; the controller is further configured to, in response to a triggering signal of the first button, control the power supply assembly to output electrical signals within a first voltage range to enable the vibration electrodes to deliver the vibrations to the user's skin; and the controller is further configured to, in response to a triggering signal of the second button, control the power supply assembly to output electrical signals within a second voltage range to enable the vibration electrodes to deliver the electric shocks to the user's skin; and the first voltage range has less voltage than the second voltage range.

In an embodiment, the first voltage range includes a range of 50 V~100 V for enabling the vibration electrodes to deliver the vibrations; and the second voltage range comprises a range of 100 V~150 V for enabling the vibration electrodes to deliver the electric shocks.

In an embodiment, the wearable device includes a Near Field Communication (NFC) chip, configured to store, receive, and transmit embedded information.

In an embodiment, the NFC chip is disposed inside the housing and near a middle top of the hollow channel.

In an embodiment, the wearable device comprises a shape of a ring and the wearable device is configured to be worn on a user's finger.

In an embodiment, a wearable system for alleviating fatigue, the system includes the wearable device for alleviating fatigue and a terminal device; the wearable device is connected by communication to the terminal device, and the terminal device includes a user interface configured for displaying one or more selected modes, receiving the user's selected modes and transmitting the selected modes to the controller; the wearable device for alleviating fatigue includes: a housing, having an inner surface forming a hollow channel; a power supply assembly, integrated inside the housing for supplying power required for operation of the device; two or more vibration electrodes, disposed on the inner surface, configured for receiving the power from the power supply assembly to deliver vibrations or electric shocks to the user's skin; a controller, coupled with the power supply assembly, for controlling the power supply assembly to output electrical signals with preset parameters based on one or more selected modes.

In an embodiment, the power supply assembly includes: a low-frequency signal generator, configured for providing a low-frequency pulse current to the vibration electrodes; and a modulation part, coupled with the low-frequency signal generator, for modulating at least one of waveform, pulse width, current, or voltage of the low-frequency pulse currents generated by the low-frequency signal generator; the controller is configured to, in response to one or more selected modes, control the modulation part to output the low-frequency pulse currents with the preset parameters to the vibration electrodes.

In an embodiment, the power supply assembly includes: a vibration motor, configured for providing mechanical vibrations to the vibration electrodes; the controller is configured to, in response to one or more selected modes, control the vibration motor to output the mechanical vibrations to the vibration electrodes.

In an embodiment, the controller further includes a first button and a second button protruding from an outer surface of the housing; the first button and the second button are oppositely disposed on the outer surface of the housing; the controller is further configured to, in response to a triggering signal of the first button, control the power supply assembly to output electrical signals within a first voltage range to enable the vibration electrodes to deliver the vibrations to the user's skin; and the controller is further configured to, in response to a triggering signal of the second button, control the power supply assembly to output electrical signals with in a second voltage range to enable the vibration electrodes to deliver the electric shocks to the user's skin; and the first voltage range has less voltage than the second voltage range.

In an embodiment, the first voltage range includes a range of 50 V~100 V for enabling the vibration electrodes to deliver the vibrations; and the second voltage range includes a range of 100 V~150 V for enabling the vibration electrodes to deliver the electric shocks.

In an embodiment, the wearable device includes a Near Field Communication (NFC) chip, configured to store, receive, and transmit embedded information.

In an embodiment, the NFC chip is disposed inside the housing and near a middle top of the hollow channel.

In an embodiment, the wearable device includes a shape of a ring and the wearable device is configured to be worn on a user's finger.

In a second aspect, in order to achieve the above objective, the present disclosure is to provide a wearable system for alleviating fatigue, including a wearable device for alleviating fatigue and a terminal device; the wearable device is connected by communication to the terminal device, and the terminal device includes a user interface configured for displaying one or more selected modes, receiving the user's selected modes and transmitting the selected modes to the controller; the wearable device for alleviating fatigue includes: a housing, having an inner surface forming a hollow channel; a power supply assembly, integrated inside the housing for supplying power required for operation of the device; two or more vibration electrodes, disposed on the inner surface, for receiving the power from the power supply assembly to deliver vibrations or electric shocks to the user's skin; and a controller, coupled with the power supply assembly, for controlling the power supply assembly to output electrical signals with preset parameters based on one or more selected modes.

In an embodiment, the power supply assembly includes:
a low-frequency signal generator, configured for providing low-frequency pulse currents to the vibration electrodes;
a modulation part, coupled with the low-frequency signal generator, for modulating at least one of waveform, pulse width, current, or voltage of the low-frequency pulse currents generated by the low-frequency signal generator; and
the controller is configured to, in response to one or more selected modes, control the modulation part to output the low-frequency pulse currents with the preset parameters to the vibration electrodes.

In an embodiment, the power supply assembly includes: a vibration motor, configured for providing mechanical vibrations to the vibration electrodes; the controller is configured to, in response to one or more selected modes, control the vibration motor to output the mechanical vibrations to the vibration electrodes.

In an embodiment, the controller further comprises a first button and a second button protruding from an outer surface of the housing; wherein the first button and the second button are oppositely disposed on the outer surface of the housing; the controller is further configured to, in response to a triggering signal of the first button, control the power supply assembly to output electrical signals within a first voltage range to enable the vibration electrodes to deliver the vibrations to the user's skin; and the controller is further configured to, in response to a triggering signal of the second button, control the power supply assembly to output electrical signals with in a second voltage range to enable the vibration electrodes to deliver the electric shocks to the user's skin; and the first voltage range has less voltage than the second voltage range.

In an embodiment, the first voltage range includes a range of 50 V~100 V for enabling the vibration electrodes to deliver the vibrations; and the second voltage range comprises a range of 100 V~150 V for enabling the vibration electrodes to deliver the electric shocks.

In an embodiment, the wearable device includes a Near Field Communication (NFC) chip, configured to store, receive, and transmit embedded information.

In an embodiment, the NFC chip is disposed inside the housing and near a middle top of the hollow channel.

In an embodiment, the wearable device includes a shape of a ring and the wearable device is configured to be worn on a user's finger.

In an embodiment, the terminal device includes a mobile terminal or an in-vehicle terminal.

The present application relates to a wearable device and systems for fatigue relief, which communicates with the terminal devices. The wearable device includes a housing, having an inner surface forming a hollow channel; a power supply assembly, integrated inside the housing for supplying power required for operation of the device; two or more vibration electrodes, disposed on the inner surface, for receiving the power from the power supply assembly to deliver vibrations or electric shocks to the user for alleviating fatigue; a controller, coupled with the power supply assembly, for controlling the power supply assembly to output electrical signals with preset parameters based on one or more selected modes. Based on one or more preset modes chosen by the user, the wearable device stimulates the vagus nerve or pain-sensing nerves in the user's skin through vibrations or electrical shocks generated by the vibration electrodes in a non-invasive manner. The above wearable device and system are aiming to prevent or alleviate the user's fatigue during work or study, thereby enhancing operational efficiency and promoting physical or mental health.

### BRIEF DESCRIPTION OF THE DRAWINGS

To more clearly illustrate the embodiments of the present application or the technical solutions in the prior art, the following briefly introduces the drawings required for describing the embodiments or the prior art. It is evident that the drawings below represent only some embodiments of the utility model. For those skilled in the art, other drawings may be derived from the structures shown in these figures without creative effort.

FIG. 1 is a structural schematic view of the wearable device for alleviating fatigue according to an embodiment of the present application.

FIG. 2 shows a circuit diagram of the vibration electrodes generating a low-frequency pulse current in the wearable device according to an embodiment of the present application.

FIG. 3 shows a circuit diagram of the vibration electrodes generating mechanical vibrations in the wearable device according to an embodiment of the present application.

FIG. 4 is a structural schematic view of the wearable system for alleviating fatigue according to an embodiment of the present application.

### DETAILED DESCRIPTION OF THE EMBODIMENTS

It should be understood that the specific embodiments described herein are only used to explain the present application and are not used to limit the present application.

The technical solutions in the embodiments according to the present application will be clearly and completely described below in conjunction with the accompanying drawings in the embodiments according to the present application. Obviously, the described embodiments are only a part of the embodiments according to the present application, and not all of the embodiments. Based on the embodiments in the present application, all other embodiments obtained by those skilled in the art without making creative labor fall within the scope of the present application.

In the embodiment of the present application, the descriptions of “first”, “second” or the like are only for descriptive purposes and cannot be understood as indicating or implying the relative importance or implicitly indicating the quantity of the technical features indicated. Therefore, features defined as “first” and “second” may explicitly or implicitly include at least one of these features. In addition, the meaning of “and/or” appearing in the entire text includes three parallel solutions, taking “A and/or B” as an example, it includes solution A, or solution B, or a solution that satisfies both A and B at the same time. In addition, the technical solutions of various embodiments can be combined with each other, but it is based on that those of ordinary skill in the art can realize the combination of technical solutions. When the combination of technical solutions is contradictory or cannot be realized, it should be considered that such combination of technical solutions does not exist and is not within the protection scope claimed by the present application.

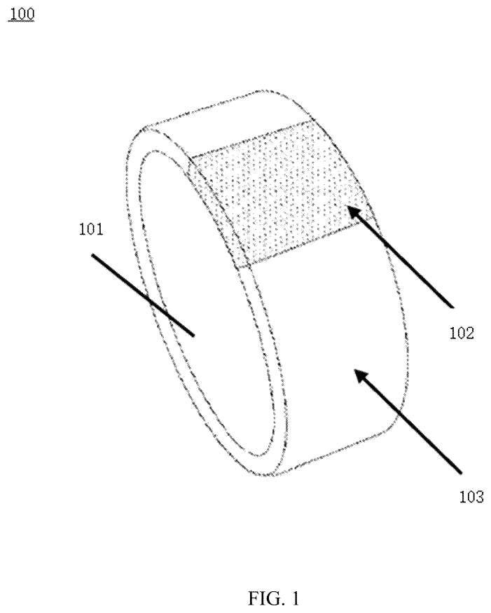

Referring to FIG. 1, FIG. 1 is a structural schematic view of the wearable device 100 for alleviating fatigue according to an embodiment of the present application.

As shown in FIG. 1, the wearable device 100 for alleviating fatigue includes a housing 102, having an inner surface forming a hollow channel; a power supply assembly 110, integrated inside the housing for supplying power required for operation of the device 100; two or more vibration electrodes 112, disposed on the inner surface, for receiving the power from the power supply assembly 110 to deliver vibrations or electric shocks to the user for alleviating fatigue; a controller 200, coupled with the power supply assembly 110, for controlling the power supply assembly 110 to output electrical signals with preset parameters based on one or more selected modes.

In an embodiment, the housing 101 is ring-shaped, and the hollow channel is designed to wear the wearable device 100 on a finger, in other words, the wearable device 100 features a hollow channel for wearing on the finger. This means that the vibration electrodes 112 are designed to be integrated with traditional accessories like rings. Therefore, the number of items a person needs to carry can be reduced, and this innovation created from the conventional tings to develop functionality that alleviates or prevents fatigue.

In an embodiment, the vibration electrodes 112 are positioned on the inner surface of the hollow channel, located on the inner side of the wearable device 100 that contacts the user's skin. At least two vibration electrodes 112 are included, which receive power from the power supply assembly 110 to deliver vibrations or electrical shocks to the user's skin. These vibrating or shocking stimulation can reach the vagus nerve or even pain-felt nerve under the skin surface, with different parameters in one or more selected modes. In some embodiments, the vibration electrodes 112 may be designed as two metal conductive plates disposed on the inner surface of the hollow channel, in a slightly curved-shape that are arranged along the curved inner surface of the ring-shaped structure. The vibration electrodes 112 are designed with a curved arc to better conform to contours of the user's finger, ensuring consistent skin contact and effective stimulation.

The housing 101 includes an inner surface that forms a hollow channel. During wear, this inner surface conforms to the user's finger size, ensuring stable contact with the vibration electrodes 112. To accommodate the variations in finger thickness across users, the inner surface of the housing 101 is equipped with an inflatable, sealed air cushion. This cushion can expand when it is inflated, allowing the ring-shaped device 100 to adapt at least one human's fingers with different sizes. An external pump (not integrated into the wearable device 100) is used to adjust the air volume based on the user's finger circumference, enabling dynamic customization for a secure fit. This design ensures consistent skin contact with the vibration electrodes 112, as well as improving the stability of low-frequency pulse currents delivered to the skin.

In some alternative embodiments, the ring-shaped wearable device 100 may be prefabricated in multiple standard sizes (e.g. large, medium, small) to accommodate varying finger thicknesses across the users.

In some alternative embodiments, the device 100 incorporates a clasp driven by a spring disposed on its inner surface. When the user inserts their finger into the hollow channel, the clasp expands by the spring action to allow the finger's entry and then contracts to secure the device 100 firmly around the finger, ensuring a stable fit across the fingers with diverse finger sizes.

It should be noted that the vibrating electrodes 112 are delivering vibrations to skin through two methods as described hereinafter:

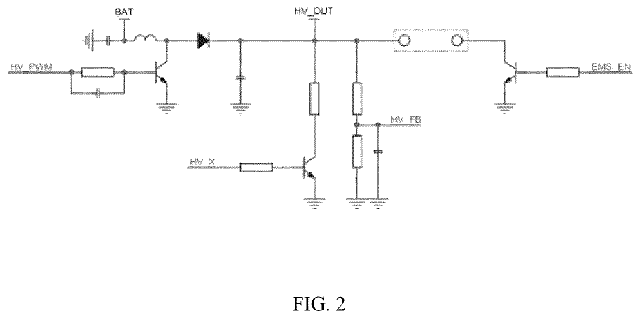

In one embodiment, the vibration electrodes 112 positioned on the inner surface of the hollow channel of the housing, acting as positive/negative electrodes for delivering low-frequency pulse currents. These electrodes 112 may deliver transcutaneous electrical nerve stimulation (TENS) and electrical muscle stimulation (EMS). As shown in FIG. 2, the two vibrating electrodes 112 work as the negative and positive electrodes to form a current loop with the skin to generate the EMS pulse current. The power supply assembly includes: a low-frequency signal generator, configured for providing low-frequency pulse current to the vibration electrodes 112; and a modulation part, coupled with the low-frequency signal generator, for modulating at least one of waveform, pulse width, current, or voltage of the pulse current generated by the low-frequency signal generator; the controller 111 is further configured to, in response to one or more selected modes, control the modulation part to output the low-frequency pulse currents with the preset parameters to the vibration electrodes 112.

In this embodiment, the controller 111 controls the output of the low-frequency pulse current from the modulation unit and the vibrating electrode units 112 to delivery vibration feeling on the user's skin, in response to one or more selected modes by the users. Specifically, the controller 111 directs the modulation unit to adjust parameters such as waveform (e.g., TENS or EMS pulses), pulse width, frequency, current/voltage amplitude, or stimulation intensity to align with the selected preset mode. Each mode enables choosing of customizable parameters which includes but doesn't limited to: different types of the pulse current waveforms, these refer to waveform types such as square, sinusoidal, or biphasic pulses, optimized for specific skin effects like TENS or EMS; different pulse widths, these refer to adjusting the duration of the pulse current; different frequencies, these refer to ranging from low-frequency (<100 Hz) to mid-frequency (100 Hz ~150 Hz) pulses; different amplitudes; different current or voltage levels.

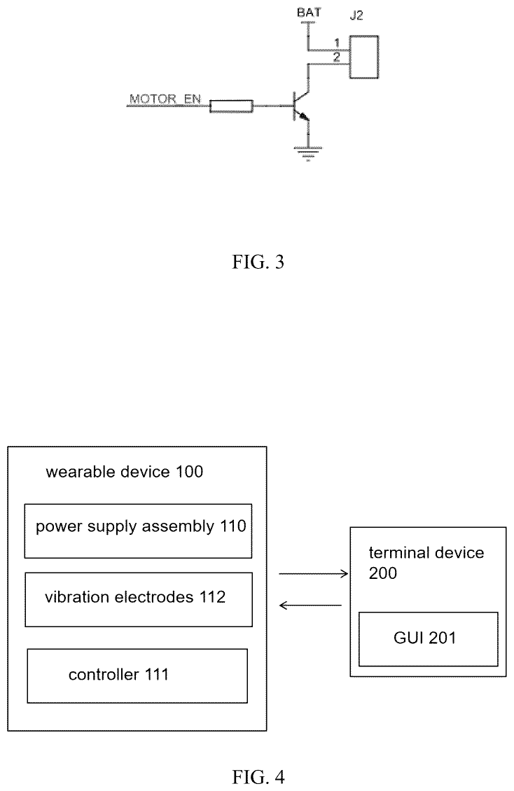

In another alternative embodiment, as shown in FIG. 3, the vibration electrodes 112 are connected to a vibration generator (a vibration motor in an exemplary embodiment) in a circuit diagram illustrating the transmission of the mechanical vibrations. The vibration electrodes 112, are positioned on the inner surface of the hollow channel of the housing 101, which delivers mechanical vibrations without requiring direct contact with the finger skin. A vibration motor to supply mechanical vibrations to the vibration electrodes 112, thus enabling non-invasive stimulation, the mechanical vibrations are used for improving blood circulation, reducing stiffness, or enhancing sensory feedback, while maintaining device ergonomics and user comfort.

Furthermore, based on one or more user selected modes, the controller 11 controls the vibration motor to output and delivery vibrations to the vibration electrodes 112 and then delivery to the user's finger. Specifically, the controller 111 can regulate the intensity (or power) of mechanical vibrations generated by the vibration motor. This is achieved by adjusting voltage or current values to modify the mechanical vibration's outputted power, thereby enabling the vibration electrodes 112 disposed on the inner surface of housing 101 to produce mechanical vibrations with varying intensities.

In one embodiment, the controller 111 further includes one or more buttons 103 protruding from the outer surface of the housing 101. These buttons 103 include a first button and a second button, which are oppositely arranged on the housing 101. Specifically, the wearable device 100 with a ring-shaped housing 101 symmetrically arranges the first button and the second button relative to the central axis of the wearable ring 100. This design allows users to intuitively control whether the vibration electrodes 112 output the mechanical vibrations or the electric shock sensation, ensuring the user's input more clear and precise during operation.

On one hand, the controller 111 is further configured to, in response to the triggering signal of the first button, control the power supply assembly 110 to output an electrical signal within a first voltage range, thereby enabling the vibration electrodes 112 to deliver vibrations to the user's skin.

In practical applications, when a user requires the vibration stimulation to prevent the fatigue, the controller 111 detects the user's activation of the first button (e.g. a single click, double click, or long press) and directs the power supply assembly 110 to output an electrical signal within the first voltage range, as such the vibration electrodes 112 can generate vibration sensation on the user's finger.

For the low-frequency pulsed current method (one of the two vibration-generation modes as aforementioned), after the power supply assembly 110 outputs the electrical signal within the first voltage range, the vibration electrodes 112 may deliver a low-frequency pulsed current to the finger skin. This results in a stimulation current at specific frequencies, causing the finger skin to perceive mild tingling, vibration, touch, pressure, or pressing sensations etc. Through extensive experimentation, the inventors of this application discovered that the power supply assembly 110 operates within a first voltage range of 50 V to 100 V and in a current of approximately 50 mA, combined with adjustments of parameters such as pulse current amplitude, frequency, pulse width, and/or duty cycle, which can induce varying intensities of vibration sensations. In contrast, in an alternative embodiment, utilizing a vibration motor to generate mechanical vibrations, the motor transmits vibrations of different frequencies to the vibration electrodes 112. In this mode, the vibration electrodes 112 cannot deliver electrical signals to the skin but instead transfers mechanical vibrations directly to the finger. This approach minimizes tingling sensations while amplifying tactile sensations such as touch, pressure, or shaking vibrations. The inventors'experiments proved that when the power supply assembly 110 operates within the same first voltage range of 50 V to 100 V and in a current of approximately 50 mA, combined with adjustments of parameters such as vibration frequency, voltage/current magnitude, as such the vibration electrodes 112 can generate mechanical vibrations with varying intensities without direct contacting the skin.

Further, the controller 111 is also configured to, in response to the triggering signal of the second button, control the power supply assembly 110 to output an electrical signal within a second voltage range to the vibration electrodes 112, enabling the vibration electrodes 112 to deliver one or more electric shocks to the finger, of which, the first voltage range is lower than the second voltage range.

In practical usage, when a user requires the electric shock stimulation to prevent the human's fatigue, the controller 111 detects the triggering of the second button (e.g. a single click, double click, or long press) and directs the power supply assembly 110 to output an electrical signal within the higher second voltage range, enabling the vibration electrodes 112 to generate the electric shock sensation. By leveraging a high-voltage, low-frequency pulsed current delivered through the vibration electrodes 112 to the finger skin, the power supply assembly 110 outputs an electrical signal within the second voltage range, inducing a high-intensity current in the skin, which results in sensations of sharp tingling, numbness, or a pronounced electric shock.

Refer to FIG. 1, in an another embodiment, the wearable device 100 further includes a Near Field Communication (NFC) chip 102, configured to store, receive, and transmit information embedded within it. In other word, this embodiment provides a wearable ring that utilizes NFC technology to store, receive, and transmit data. Specifically, the wearable device 100 integrates the NFC chip 102, which is designed to manage embedded information through wireless communication within short-range proximity.

In the existing technologies, Near Field Communication (NFC) is primarily used as a digital key for locking or unlocking access systems (e.g. cars or homes) or as a mobile-embedded solution for reading short-data tags to enable payment transactions.

This embodiment provides a wearable device 100 that integrates the function of an access key for vehicles or buildings, with the function of fatigue-alleviation described in the aforementioned embodiments. Designed as a ring shape, this wearable device 100 not only mitigates fatigue during work or study but also serves as a versatile access key. It is particularly advantageous for car owners: during the driving, the wearable ring 100 can both prevent or reduce driver fatigue and act as a car access key, significantly minimizing the number of items a driver must carry and achieving the multi-purposes.

It should be noted that the Near Field Communication (NFC) chip 102 in this application is integrated within the housing 101 of the wearable device 100 and is electrically connected to the built-in power supply assembly 110 of the housing 101 to provide energy to the NFC chip 102. As a result, the wearable ring in this application functions as an active NFC-enabled smart ring.

In practical usage, the users only need to tap their fingers near an access point (e.g., a car or door lock) to perform locking or unlocking operations. There is no need for deliberate movement of the smart ring toward the access point, as the signal is automatically transmitted from the ring when the user's finger naturally moves in relation to the hand or the wrist.

To enhance the stability of signal transmission between the NFC chip in the wearable ring 100 and external terminal devices (e.g. cars or access systems) 200, the NFC chip 102 is positioned within the housing 101 of the wearable device 100 and located at the top of the hollow channel to optimize alignment during the aforementioned locking/unlocking operations.

Further, through rigorous testing by the inventors, the effective read/write distance of the wearable smart ring is determined to be 6 cm. When the user's finger brings the ring less than 6 cm from the target terminal device (e.g., a door access point), the NFC chip 102 can reliably exchange data with receivers, transmitters, or transceivers. In addition to the NFC chip and power supply assembly 110, the wearable smart ring includes a t least one capacitors for signal tuning, ensuring optimal performance during data transmission by minimizing interference and enhancing signal integrity.

In some embodiments, the top of the housing 101 protrudes a display screen that exhibits real-time information such as time, remaining battery level, and charging status.

As shown in FIG. 4, the present application further discloses a wearable system for fatigue alleviation, including the aforementioned wearable device 100 for fatigue alleviation and a terminal device 200. The wearable device 100 is connected by communication to the terminal device 200, which includes a user interface (UI) 201 configured to display one or more selected modes, then the selected mode is transmitted to the controller 111 of the wearable device 100 for execution. In one embodiment, the terminal device 200 may be a mobile terminal (e.g., smartphone, tablet) or an in-vehicle terminal (e.g., car information system). The user interface 201 on the terminal device 200 is configured to receive the user's inputs and includes at least one display screen, such as a liquid crystal display (LCD), light-emitting diode (LED) display, or organic LED (OLED) display. The display screen may also incorporate a touchscreen interface for interactive control. The user interface 201 is connected by communication to the control unit 111 embedded within the wearable device 100 to enable bidirectional data exchange and activation of the selected mode.

Understandable, prior to using the wearable device 200, the users can set and define one or more selected modes beforehand, which may include: selecting a value from preset voltage or current clusters, such as choosing a specific voltage from the first voltage range or second voltage range; or choosing a value from preset frequency clusters (e.g., 50 Hz for pain relief or 100 Hz for vibration); setting the start time and duration of vibrations or electrical shock stimulation and so on. The controller 111 communicates with the terminal device (e.g., smartphone or in-vehicle system) and receives the user's selected parameters to execute the corresponding operations.

A wearable device and system for alleviating fatigue are disclosed, the device connected to a terminal device includes: a housing, having an inner surface forming a hollow channel; a power supply assembly, integrated inside the housing, for supplying power for the device's operation; two or more vibration electrodes, disposed on the inner surface, for receiving the power from the power supply assembly to deliver vibrations or electric shocks to the user's skin; and a controller, coupled with the power supply assembly, for controlling the power supply assembly to output electrical signals with preset parameters based on one or more selected modes. Under one or more selected modes chosen by the user, the wearable device delivers vibrations or electric shocks to the user's skin through the vibration electrodes, stimulating the vagus nerve or even the pain nerve in a non-invasive manner, to prevent or alleviate fatigue during work and study.

In the present application, the terms “include”, “comprise” or any other variation thereof are intended to cover a non-exclusive inclusion, such that a process, method, article or system that includes a list of elements not only includes those elements, but also includes other elements not expressly listed, or also includes elements inherent to the process, method, article or system. In a case of without further restrictions, an element defined by the statement “includes . . . ” does not exclude the existence of other identical elements in a process, method, article or system that includes this element.

The serial numbers of the embodiments of the present application are only for description and do not represent the advantages and disadvantages of the embodiments. By the description of the above implementation mode, those skilled in the art can clearly understand that the above embodiment method can be implemented by means of software plus the necessary general hardware platform mode. Certainly, it can also be implemented by hardware, but in many cases the former is a better implementation mode. Based on this understanding, the technical solution of the present application, or the part that contributes to the prior art, can be embodied in the form of a software product, which is stored in a storage medium (such as a read-only memory/random access memory, a disk, or an optical disk), and includes several instructions for enabling a terminal device (which can be a mobile phone, a computer, a server, or a network device, etc.) to execute the methods of each embodiment of the present application. The above are only some embodiments of the present application, and do not limit the scope of the present application. Any equivalent structure or equivalent process transformation made using the contents of the specification and drawings of the present application, or directly or indirectly used in other related technical fields, are also included in the scope of the present application.

What is claimed is:

1. A wearable device for alleviating fatigue, connected by communication to a terminal device, comprising:
a housing, having an inner surface forming a hollow channel;
a power supply assembly, integrated inside the housing for supplying power required for operation of the device;
two or more vibration electrodes, disposed on the inner surface, configured for receiving the power from the power supply assembly to deliver vibrations or electric shocks to the user for alleviating fatigue; and
a controller, coupled with the power supply assembly, for controlling the power supply assembly to output electrical signals with preset parameters based on one or more selected modes.

2. The wearable device according to claim 1, wherein the power supply assembly comprises:
a low-frequency signal generator, configured for providing low-frequency pulse currents to the vibration electrodes; and
a modulation part, coupled with the low-frequency signal generator, for modulating at least one of waveform, pulse width, current, or voltage of the low-frequency pulse current generated by the low-frequency signal generator;
wherein the controller is further configured to, in response to one or more selected modes, control the modulation part to output the low-frequency pulse currents with the preset parameters to the vibration electrodes.

3. The wearable device according to claim 1, wherein the power supply assembly comprises a vibration motor, configured for providing mechanical vibrations to the vibration electrodes; the controller is further configured to, in response to one or more selected modes, control the vibration motor to output the mechanical vibrations to the vibration electrodes.

4. The wearable device according to claim 1, wherein the controller further comprises a first button and a second button protruding from an outer surface of the housing; the first button and the second button are oppositely disposed on the outer surface of the housing;
the controller is further configured to, in response to a triggering signal of the first button, control the power supply assembly to output electrical signals within a first voltage range to enable the vibration electrodes to deliver the vibrations to the user's skin;
the controller is further configured to, in response to a triggering signal of the second button, control the power supply assembly to output electrical signals within a second voltage range to enable the vibration electrodes to deliver the electric shocks to the user's skin; and
the first voltage range has less voltage than the second voltage range.

5. The wearable device according to claim 4, wherein the first voltage range comprises a range of 50 V~100 V for enabling the vibration electrodes to deliver the vibrations; and
the second voltage range comprises a range of 100 V~150 V for enabling the vibration electrodes to deliver the electric shocks.

6. The wearable device according to claim 1, wherein the wearable device comprises a Near Field Communication (NFC) chip, configured to store, receive, and transmit embedded information.

7. The wearable device according to claim 6, wherein the NFC chip is disposed inside the housing and near a middle top of the hollow channel.

8. The wearable device according to claim 1, wherein the wearable device comprises a shape of a ring and the wearable device is configured to be worn on a user's finger.

9. A wearable system for alleviating fatigue, wherein the system comprises a wearable device for alleviating fatigue and a terminal device; the wearable device is connected by communication to the terminal device, and the terminal device comprises a user interface configured for displaying one or more selected modes, receiving the user's selected modes and transmitting the selected modes to the controller;
wherein the wearable device for alleviating fatigue comprises:
a housing, having an inner surface forming a hollow channel;
a power supply assembly, integrated inside the housing for supplying power required for operation of the device;
two or more vibration electrodes, disposed on the inner surface, configured for receiving the power from the power supply assembly to deliver vibrations or electric shocks to the user's skin; and
a controller, coupled with the power supply assembly, for controlling the power supply assembly to output electrical signals with preset parameters based on one or more selected modes.

10. The wearable system according to claim 9, wherein the power supply assembly comprises:
a low-frequency signal generator, configured for providing low-frequency pulse currents to the vibration electrodes; and
a modulation part, coupled with the low-frequency signal generator, for modulating at least one of waveform, pulse width, current, or voltage of the low-frequency pulse currents generated by the low-frequency signal generator;
wherein the controller is configured to, in response to one or more selected modes, control the modulation part to output the low-frequency pulse currents with the preset parameters to the vibration electrodes.

11. The wearable system according to claim 9, wherein the power supply assembly comprises: a vibration motor, configured for providing mechanical vibrations to the vibration electrodes;
wherein the controller is configured to, in response to one or more selected modes, control the vibration motor to output the mechanical vibrations to the vibration electrodes.

12. The wearable system according to claim 9, wherein the controller further comprises a first button and a second button protruding from an outer surface of the housing; the first button and the second button are oppositely disposed on the outer surface of the housing; the controller is further configured to, in response to a triggering signal of the first button, control the power supply assembly to output electrical signals within a first voltage range to enable the vibration electrodes to deliver the vibrations to the user's skin;
the controller is further configured to, in response to a triggering signal of the second button, control the power supply assembly to output electrical signals with in a second voltage range to enable the vibration electrodes to deliver the electric shocks to the user's skin; and
the first voltage range has less voltage than the second voltage range.

13. The wearable system according to claim 12, wherein the first voltage range comprises a range of 50 V~100 V for enabling the vibration electrodes to deliver the vibrations; and the second voltage range comprises a range of 100 V~150 V for enabling the vibration electrodes to deliver the electric shocks.

14. The wearable system according to claim 9, wherein the wearable device comprises a Near Field Communication (NFC) chip, configured to store, receive, and transmit embedded information.

15. The wearable system according to claim 14, wherein the NFC chip is disposed inside the housing and near a middle top of the hollow channel.

16. The wearable system according to claim 9, wherein the wearable device comprises a shape of a ring and the wearable device is configured to be worn on a user's finger.

17. The wearable system according to claim 9, wherein the terminal device comprises a mobile terminal or an in-vehicle terminal.
